(19) United States
(12) Patent Application Publication
(10) Pub. No.: US 2023/0398105 A1
(43) Pub. Date: Dec. 14, 2023

(54) TREATMENT REGIMENS FOR PARKINSON'S DISEASE

(71) Applicant: BIAL - PORTELA & Cª, S.A., S. Mamede do Coronado (PT)

(72) Inventors: Patricio Manuel Vieira Araújo Soares Da Silva, S. Mamede do Coronado (PT); José Francisco Da Costa De Pinho Rocha, S. Mamede do Coronado (PT)

(21) Appl. No.: 18/032,034

(22) PCT Filed: Oct. 14, 2021
(86) PCT No.: PCT/PT2021/050036

§ 371 Date: Apr. 14, 2023

(30) Foreign Application Priority Data
Oct. 16, 2020 (GB) 2016425.7

(51) Int. Cl. A61K 31/4439; A61K 31/198; A61K 31/165; A61P 25/16

(52) U.S. Cl. CPC A61K 31/4439; A61K 31/198; A61K 31/165; A61P 25/16

(57) ABSTRACT

Opicapone, or a pharmaceutically acceptable derivative thereof, in combination with levodopa, or a pharmaceutically acceptable derivative thereof, for use in treating the symptoms of Parkinson's disease in a patient suffering from unpredictable motor fluctuations.

### FIELD OF THE INVENTION

This invention relates to the treatment of unpredictable motor fluctuations in Parkinson's disease. In particular, the invention relates to the use of opicapone, or a pharmaceutically acceptable derivative thereof, in combination with levodopa, or a pharmaceutically acceptable derivative thereof, in treating the symptoms of Parkinson's disease in a patient suffering from unpredictable motor fluctuations.

### BACKGROUND OF THE INVENTION

Levodopa (L-DOPA) has been used in clinical practice for several decades in the symptomatic treatment of various conditions, including Parkinson's disease. Levodopa is able to cross the blood-brain barrier, where it is then converted to dopamine by the enzyme DOPA decarboxylase (DDC), thus increasing dopamine levels in the brain. However, conversion of levodopa to dopamine may also occur in peripheral tissues, possibly causing adverse effects. Therefore, it has become standard clinical practice to co-administer a peripheral DDC inhibitor (DDCI), such as carbidopa or benserazide, as adjunctive therapies. DDCIs prevent conversion of levodopa to dopamine in peripheral tissues. Levodopa therapy remains the most effective treatment for the management of Parkinson's disease (Ferreira J, et al., Eur. J. Neurol., 2013; 20, 5-15).

During the earlier stages of Parkinson's disease, levodopa therapy can almost entirely suppress symptoms of Parkinson's disease until the next dose is administered. However, most patients receiving long-term levodopa therapy will develop motor complications, such as end-of-dose motor fluctuations and dyskinesia, at more advanced stages of Parkinson's disease (Aquino C C, Fox S H, Mov. Disord., 2015, 30, 80-89). Patients often report spending several hours per day with end-of-dose motor fluctuations in the so-called “off” state and this can have a substantial effect on their quality of life (Chapuis S, Ouchchane L, Metz O, Gerbaud L, Durif et al., Mov. Disord. 2005, 20, 224-30). The development of motor complications, such as end-of-dose motor fluctuations, defines the transition from the early stage of Parkinson's disease to a more advanced stage of the disease. As such, the control of motor complications eventually becomes a key clinical need for almost all patients (Poewe W, Neurology, 2009, 72, S65-73).

End-of-dose motor fluctuations are linked to the short half-life of oral levodopa (about 60-90 min). Catechol-O-methyltransferase (COMT) inhibitors increase the plasma elimination half-life of levodopa and decrease peak-trough variations and provide clinical improvements in Parkinson's disease patients afflicted with end-of-dose motor fluctuations.

2,5-dichloro-3-[5-(3,4-dihydroxy-5-nitrophenyl)-1,2,4-oxadiazol-3-yl]-4,6-dimethylpyridine 1-oxide (opicapone) is a potent and long-acting COMT inhibitor that reduces the degradation of levodopa to the inactive metabolite 3-O-methyldopa. Opicapone is bioactive, bioavailable and exhibits low toxicity. Thus, opicapone has potentially valuable pharmaceutical properties in the treatment of some central and peripheral nervous system disorders where inhibition of COMT may be of therapeutic benefit, such as, for example, mood disorders; movement disorders, such as Parkinson's disease, parkinsonian disorders and restless legs syndrome; gastrointestinal disturbances; oedema formation states; and hypertension.

Further research has focused on optimising opicapone into a stable and bioavailable form. For example, WO 2009/116882 describes various polymorphs of opicapone, with polymorph A being both kinetically and thermodynamically stable. WO 2010/114404 and WO 2010/114405 describe stable opicapone formulations used in clinical trials. WO 2013/089573 describes optimised methods for producing opicapone using simple starting materials and with good yields. The development of opicapone is described in L. E. Kiss et al, J. Med. Chem., 2010, 53, 3396-3411 and it was approved, in combination with levodopa and a DCCI, for the treatment of Parkinson's disease in the EU in June 2016, the US in April 2020 and Japan in June 2020 under the tradename “Ongentys”.

In all cases, opicapone is licenced as an adjuvant therapy to levodopa/DDCI preparations for use in patients experiencing motor fluctuations. For example, the European label states: “Ongentys is indicated as adjunctive therapy to preparations of levodopa/DOPA decarboxylase inhibitors (DDCI) in adult patients with Parkinson's disease and end-of-dose motor fluctuations who cannot be stabilised on those combinations” (emphasis added). The US label states: “ONGENTYS is a catechol-O-methyltransferase (COMT) inhibitor indicated as adjunctive treatment to levodopa/carbidopa inpatients with Parkinson's disease (PD) experiencing “off” episodes” (emphasis added).

The licencing of opicapone is based on the primary results from two pivotal phase III trials of opicapone in patients beyond the early stages of Parkinson's disease (i.e. in patients experiencing end-of-dose motor fluctuations). The trials are known as BIPARK-I (Ferreira et al., Lancet Neurol., 2016, 15, 154-65) and BIPARK-II (Lees et al., JAMA Neurol., 2017, 74, 197-206).

BIPARK-I demonstrated opicapone was superior to a placebo combined with levodopa/DCCI and non-inferior to previously-licenced COMT inhibitor, entacapone, in terms of its ability to reduce the time patients spent in the “off” state. BIPARK-II confirmed opicapone's efficacy and safety. These pivotal phase III trials confirmed the provisional results from smaller phase II trials. Post hoc analysis of the combined BIPARK studies suggests that opicapone also slows the rate of increase of time patients spend in the “off” state. In other words, opicapone appears to slow the progression of Parkinson's disease with respect to the levodopa need in patients at more advanced stages of Parkinson's disease (WO 2016/083875), i.e. in patients experiencing end-of-dose motor fluctuations.

Motor complications occur in about 50% of Parkinson's disease patients within 5 years of levodopa treatment and are associated with significant quality of life (QoL) deterioration (Dodel R., Berger K., Oertel W., PharmacoEconomics, 2001, 19, 1013-38). A subgroup of Parkinson's disease patients can also experience unpredictable motor fluctuations, i.e. a worsening of parkinsonian symptoms unrelated to the timing of medication, with symptoms often coming on over a few seconds (Aquino C., Fox S., J. Mov. Disord., 2015, 30, 80-9). These patients can experience severe on/off fluctuations, presenting as very rapid transition from the “on” state to the “off” state (Aquino C., Fox S., J. Mov. Disord., 2015, 30, 80-9). This stage is sometimes described as the “complicated stage” of Parkinson's disease (Carrarini C. et al., Biomolecules, 2019, 9, 388) and is particularly difficult to treat. Indeed, as highlighted in the European Federation of Neurological Societies (EFNS) recommendations on Parkinson's disease treatment, patients with unpredictable on-off were either not included or constituted <5% of the total population. Conversely, such severe motor fluctuations often force clinicians to turn to device-aided therapies, namely deep brain stimulation, subcutaneous apomorphine infusion and levodopa/carbidopa intestinal gel that are usually effective in consistently reducing the absolute off-time per day, though no specific sub-analysis on patients with unpredictable motor fluctuations have been published (Katzenschlager R. et al. Lancet. Neurol., 2018, 17, 749-759; Olanow C., Lancet. Neurol., 2014, 13, 141-9; Deuschl G., New Eng. J. Med., 2006, 355, 896-908). However, device-aided therapies are considered the treatment of choice if oral drugs do not adequately control motor fluctuations.

To date, the treatment of unpredictable motor fluctuations remains very challenging as there are no evidence that a specific adjunctive therapy is effective for Parkinson's disease patients with such severe motor complications.

Therefore, there remains a need for levodopa treatment regimens that are effective in the treatment of patients with unpredictable motor fluctuations. In particular, there remains a need for effective levodopa treatment regimens that can improve acute symptoms of unpredictable motor fluctuations in Parkinson's disease, preferably without causing further side effects and/or treatment-emergent adverse events.

### SUMMARY OF THE INVENTION

The present inventors pooled the results of two randomized double-blind clinical trials (BIPARK-I and BIPARK-II) and stratified patients on the presence or absence of unpredictable motor fluctuations by means of the UPDRS section IV. As described in Section D, below, among 278 Parkinson's disease patients with unpredictable motor fluctuations (87 on 50 mg, 98 on 25 mg, 93 on placebo), both opicapone (25 mg) and opicapone (50 mg) were effective in reducing motor complications. Surprisingly, opicapone was more effective in patients with unpredictable motor fluctuations is spite of these patients being considered more difficult to treat.

Accordingly, in a first general embodiment, the invention provides opicapone, or a pharmaceutically acceptable derivative thereof, in combination with levodopa, or a pharmaceutically acceptable derivative thereof, for use in treating the symptoms of Parkinson's disease in a patient suffering from unpredictable motor fluctuations.

In a second general embodiment, the invention provides a method of treating the symptoms of Parkinson's disease in a patient suffering from unpredictable motor fluctuations comprising (optionally diagnosing the patient as suffering from unpredictable motor fluctuations and subsequently) administering to the patient a therapeutically effective amount of opicapone, or a pharmaceutically acceptable derivative thereof, in combination with levodopa, or a pharmaceutically acceptable derivative thereof.

In a third general embodiment, the invention provides the use of opicapone, or a pharmaceutically acceptable derivative thereof, in combination with levodopa, or a pharmaceutically acceptable derivative thereof, for the manufacture of a medicament for the treatment of the symptoms of Parkinson's disease in a patient suffering from unpredictable motor fluctuations.

In a fourth general embodiment, the invention provides the use of opicapone, or a pharmaceutically acceptable derivative thereof, in combination with levodopa, or a pharmaceutically acceptable derivative thereof, for the manufacture of a medicament for the treatment of unpredictable motor fluctuations in a patient suffering from Parkinson's disease.

### BRIEF DESCRIPTION OF THE DRAWINGS

The invention will now be described in detail with reference to the accompanying drawings and tables, in which:

FIG. 1 shows the reduction in absolute off-time after 14-15 weeks of treatment with opicapone (25 mg) and opicapone (50 mg) compared to placebo in patients suffering from unpredictable motor fluctuations and those not suffering from unpredictable motor fluctuations. LSM=Least Square Mean; LCL=Lower Confidence Limit; UCL=Upper Confidence Limit; N=number of patients.

### DETAILED DESCRIPTION OF THE INVENTION

### A. Definitions

The following definitions apply to the terms used throughout this specification, unless otherwise limited in specific instances.

The term “idiopathic Parkinson's disease” encompasses most (80-85%) Parkinson's disease. It typically involves prominent bradykinesia and variable associated extrapyramidal signs and symptoms. It is accompanied by degeneration of the nigrostriatal dopaminergic system, with neuronal loss and reactive gliosis in the substantia nigra found at autopsy. In idiopathic Parkinson's disease, α-synuclein typically accumulates in neuronal perikarya (Lewy bodies) and neuronal processes (Lewy neurites). Idiopathic Parkinson's excludes drug-induced parkinsonism, vascular parkinsonism, normal pressure hydrocephalus, corticobasal degeneration, progressive supranuclear palsy and multiple system atrophy.

The term “early idiopathic Parkinson's disease” or “early Parkinson's disease” refers to the early stage of the disease, when overt symptoms allow a diagnosis of idiopathic Parkinson's disease (according to either the United Kingdom Parkinson's Disease Society Brain Bank Clinical Diagnostic Criteria or the Movement Disorder Society criteria) but those symptoms are mild and unilateral with a complete response to treatment being possible. In particular, this patient group's Parkinson's disease is treatable (i.e. their symptoms can be controlled) with preparations of levodopa and a DDCI without motor complications, such as end-of-dose motor fluctuations and/or dyskinesia.

The term “symptoms of Parkinson's disease” includes both motor symptoms (e.g. tremor, rigidity, bradykinesia and postural instability) and non-motor symptoms (e.g. cognitive changes, gastrointestinal symptoms, loss of sight, taste and/or smell, pain, fatigue, light-headedness, sexual problems, sleep disorders and weight loss). Such symptoms can be assessed using one or more of the symptomatic readouts known in the art, especially those specifically mentioned herein.

The term “motor complications” relates to Parkinson's disease symptoms which are a consequence of chronic treatment, namely levodopa treatment, comprised in motor fluctuations (including wearing-off phenomenon), levodopa-induced dyskinesia and other disease-related features not present at the early stage of disease. They arise when levodopa therapy alone no longer provides complete control of the patient's symptoms. They include motor fluctuations and/or dyskinesia. Motor complications are sustained, but not necessarily regular or predictable, such that they quantifiably and negatively impact on the patient's quality of life (QoL). Motor complications can overlap with motor symptoms of Parkinson's disease. However, a motor symptom which is initially treatable by levodopa therapy, but which re-emerges at a later stage of disease in spite of maintaining levodopa therapy, is considered a motor complication.

The term “motor fluctuations” includes end-of-dose fluctuations, paradoxical fluctuations and unpredictable on/off.

The term ““off” period” also known as ““off” episodes” is defined as the times during which a patient treated with levodopa no longer experiences its symptomatic benefit and is said to be in an “off” state. On the other hand, when a patient treated with levodopa experiences its symptomatic benefit, the patient is in an “on” state during an ““on” period”.

The term “absolute off-time” is the combined total daily of 30-minute periods spent in an “off” state. Conversely, the term “absolute on-time” is the combined total daily of 30-minute periods spent in an “on” state. Periods where the patient is asleep are excluded from either group.

The term “end-of-dose motor fluctuations”, also known as the “wearing off” phenomenon, relates to the predictable re-emergence or worsening of symptoms before administration of the next dose of levodopa therapy. Typically, they start 3-4 hours after a dose of levodopa, as the medication wears off and symptoms re-emerge or worsen. Symptoms then typically improve 15-45 minutes after the next levodopa dose is taken.

The term “unpredictable motor fluctuations”, also known as “unpredictable on/off fluctuations” or “on/off phenomenon”, relates to the unpredictable re-emergence or worsening of symptoms at any time, such as severe troublesome dyskinesias, severe/unpredictable end-of-dose motor fluctuations, painful “off” dystonia and morning akinesia (Fabbri M. et al., Mov. Disord., 2018, 33, 1528-1539). Unpredictable motor fluctuations are unrelated to the timing of the next dose and may occur at any time during the day. However, re-emergence or worsening of symptoms close to a dose of levodopa therapy can be unpredictable if they do not occur regularly. Within the clinical trials analysed, unpredictable on/off fluctuations were defined as present if clinicians scored “NO” at the UPDRS item 36 (“Are “off” periods predictable?”), or “YES” in at least one of the following UPDRS items: 37 (“Are “off” periods unpredictable?”) and 38 (“Do “off” periods come on suddenly, within a few seconds?”).

The term “dyskinesia” or “levodopa-induced dyskinesias” includes peak dose dyskinesia, diphasic dyskinesia and “off” dyskinesia. Common symptoms include chorea and dystonia. Less common symptoms include akathasia (excessive motor restlessness), a high stepped overshooting gait, rapid alternating movements (RAM) of legs, blepharospasm, and mixed pattern of abnormal movements (Fahn S., Ann. Neurol., 2000, 47, S2-S9).

The term “responders” is defined as patients achieving at least a 1-hour reduction in absolute off-time and/or the proportions of patients achieving at least a 1-hour increase in absolute on-time at the end of the double-blind phase of the trial.

The term “adjunctive therapy”, also known as adjunct therapy, add-on therapy, or adjuvant care, is therapy that is given in addition to the primary or initial therapy to maximize its effectiveness. In the current application, levodopa is the primary therapy and the DCCI and COMT inhibitor (i.e. opicapone) are the adjunctive therapies.

The term “treatment-emergent adverse events” is defined as any event not present before exposure to the study drug or any event already present that worsens in either intensity or frequency after first intake of study drug until 2 weeks after last intake of the study drug.

### B. Opicapone Efficacy in Parkinson's Disease Patients with Unpredictable Motor Fluctuations

The invention provides opicapone, or a pharmaceutically acceptable derivative thereof, in combination with levodopa, or a pharmaceutically acceptable derivative thereof, for use in treating the symptoms of Parkinson's disease in a patient suffering from unpredictable motor fluctuations.

The invention also provides a method of treating the symptoms of Parkinson's disease in a patient suffering from unpredictable motor fluctuations comprising (optionally diagnosing the patient as suffering from unpredictable motor fluctuations and subsequently) administering to the patient a therapeutically effective amount of opicapone, or a pharmaceutically acceptable derivative thereof, in combination with levodopa, or a pharmaceutically acceptable derivative thereof.

The invention also provides use of opicapone, or a pharmaceutically acceptable derivative thereof, in combination with levodopa, or a pharmaceutically acceptable derivative thereof, for the manufacture of a medicament for the treatment of the symptoms of Parkinson's disease in a patient suffering from unpredictable motor fluctuations.

The invention also provides use of opicapone, or a pharmaceutically acceptable derivative thereof, in combination with levodopa, or a pharmaceutically acceptable derivative thereof, for the manufacture of a medicament for the treatment of unpredictable motor fluctuations in a patient suffering from Parkinson's disease.

### Opicapone Dosage and Regimens

The trial analysis described in Section D, below, confirms that both opicapone (25 mg) and opicapone (50 mg) were surprisingly effective in reducing motor complications in patients with unpredictable motor fluctuations. In a preferred embodiment, the opicapone, or a pharmaceutically acceptable derivative thereof, is administered once daily at a dose equivalent to 10 to 100 mg of opicapone, preferably equivalent to 25 to 50 mg of opicapone, more preferably equivalent to about 50 mg of opicapone.

Although a 50 mg dose of opicapone is most preferred in terms of efficacy, the results in Section D, below, confirm that in patients with unpredictable motor fluctuations, opicapone (25 mg) is surprisingly still effective and its use reduces drug intake, with related benefits in cost and possible side effects. Therefore, an opicapone dose of about 25 mg is still highly preferred.

In a generally preferred embodiment, opicapone is administered in its non-derivative (e.g. non-salt) form.

Patients with Parkinson's disease suffering from unpredictable motor fluctuations tend to require more frequent dosing with levodopa and/or larger doses of levodopa. Inclusion of opicapone as an adjuvant therapeutic might decrease the required dose of levodopa. In a preferred embodiment, the levodopa, or a pharmaceutically acceptable derivative thereof, is administered 3 to 10 times per day. In a more preferred embodiment, multiple doses of levodopa are administered orally in the form of a tablet or capsule.

The dosages of levodopa can be varied to suit the needs of the patient. In another preferred embodiment, that is complementary to the dosing frequency, the levodopa, or a pharmaceutically acceptable derivative thereof, is administered at a total daily dose equivalent to 300 to 2000 mg of levodopa, preferably equivalent to 500 to 1000 mg of levodopa. In particular, the levodopa, or a pharmaceutically acceptable derivative thereof, is administered 3 to 10 times per day at a total daily dose equivalent to 300 to 2000 mg of levodopa, preferably equivalent to 500 to 1000 mg of levodopa.

Patients suffering from the later stages of Parkinson's disease and treated with levodopa have been found to benefit from modified forms of levodopa. In an embodiment, the patient is treated with a deuterated form of levodopa (e.g., those disclosed in (WO 2017/060870)) or modified-release levodopa, such as prolonged-release levodopa, controlled-released levodopa, extended-release levodopa, modified-release levodopa or levodopa intestinal gel.

The combination of a deuterated form of levodopa and opicapone has been shown to reduce dyskinesia in a 6-OH-L-DOPA-induced parkinsonism model (WO 2017/060870). Such models are poor predictors of clinical efficacy in idiopathic Parkinson's disease. Furthermore, deuterated levodopa is expensive. Therefore, a preferred embodiment relates to opicapone, or a pharmaceutically acceptable derivative thereof, in combination with levodopa, or a pharmaceutically acceptable derivative thereof, for use in treating the symptoms of Parkinson's disease in a patient suffering from unpredictable motor fluctuations, wherein the levodopa is not deuterated levodopa.

Intestinal gels have been shown to be effective in treating later stages of Parkinson's disease, but require administration with a pump directly into the intestine. As opicapone has been found to be particularly effective in treating patients suffering from unpredictable motor fluctuations, it could considerably delay the need for intestinal gels. Therefore, a preferred embodiment relates to opicapone, or a pharmaceutically acceptable derivative thereof, in combination with levodopa, or a pharmaceutically acceptable derivative thereof, for use in treating the symptoms of Parkinson's disease in a patient suffering from unpredictable motor fluctuations, wherein the levodopa is not administered in the form of a levodopa intestinal gel. More preferably, the levodopa is in the form of a tablet or capsule.

More preferably, levodopa is neither deuterated nor in the form of a levodopa intestinal gel.

Levodopa has been found to be more bioavailable when administered with a DDCI. In a preferred embodiment, the combination of opicapone, or a pharmaceutically acceptable derivative thereof, and levodopa, or a pharmaceutically acceptable derivative thereof, further comprises a DDCI. In a more preferred embodiment, the DDCI is carbidopa or benserazide. In another more preferred embodiment, the DDCI is administered 3 to 10 times per day.

The dosages of the DDCI can be varied to suit the needs of the patient. In another more preferred embodiment, that is complementary to the dosing frequency, the DDCI is administered at a total daily dose of 25 to 500 mg, preferably 75 to 250 mg. In particular, carbidopa or benserazide is administered 3 to 10 times per day at a total daily dose of 25 to 500 mg, preferably 75 to 250 mg.

To increase patient compliance, levodopa is often administered at the same time as the DDCI meaning they are administered the same number of times per day. In another preferred embodiment when a DDCI is combined with opicapone, or a pharmaceutically acceptable derivative thereof, and levodopa, or a pharmaceutically acceptable derivative thereof, the levodopa, or a pharmaceutically acceptable derivative thereof, and the DDCI are administered in a single dosage unit. In a more preferred embodiment, the levodopa, or a pharmaceutically acceptable derivative thereof, and the DDCI are administered in a single dosage unit in the form of a capsule or tablet.

In contrast to levodopa, DDCIs and other COMT inhibitors (e.g., entacapone), opicapone is a long-acting COMT inhibitor. In a preferred embodiment, the opicapone is administered once daily or once weekly, preferably once daily.

Opicapone can interact with levodopa. In a preferred embodiment, the opicapone is administered more than 1 hour before or after administration of levodopa. More preferably, opicapone is administered more than 1 hour before or after the last daily dose of levodopa.

Opicapone can interact with food. In a preferred embodiment, the opicapone is administered more than 1 hour before or after a meal.

In a more preferred embodiment, the opicapone is administered at or near to bedtime.

Opicapone shows good tolerability and a low incidence of adverse events (AEs) including treatment-emergent adverse events, so can be administered to a patient over extended periods without continuous assessment of toxicity. In a preferred embodiment, the treatment lasts at least 10 weeks, preferably at least 15 weeks.

### Patient Population

The trial analysis described in Section D, below, confirms that opicapone is surprisingly effective in reducing motor complications in patients with unpredictable motor fluctuations, as defined if clinicians scored “NO” at the UPDRS item 36 (“Are “off” periods predictable?”), or “YES” at least in one of the following UPDRS items: 37 (“Are “off” periods unpredictable?”) and 38 (“Do “off” periods come on suddenly, within a few seconds?”).

Therefore, in a preferred embodiment, the patient suffering from unpredictable motor fluctuations is one who would score “NO” at the UPDRS item 36 and/or “YES” in at least one of the UPDRS items 37 or 38.

In a preferred embodiment, the patient suffering from unpredictable motor fluctuations suffers from sudden and random changes from an “on” state to a marked “off” state over less than 10 minutes, preferably less than 5 minutes, more preferably less than 2 minutes, even more preferably less than 1 minute, with no apparent relationship to the timing of levodopa administration.

The skilled person is aware that whilst unpredictable motor fluctuations often occur suddenly or randomly, they might not occur instantly. Therefore, in a more preferred embodiment, the patient suffering from unpredictable motor fluctuations suffers from sudden and random changes from an “on” state to a marked “off” state over 1 second to 10 minutes, preferably 5 seconds to 5 minutes, more preferably 10 seconds to 2 minutes, even more preferably 30 seconds to 1 minute, with no apparent relationship to the timing of levodopa administration.

As defined above, re-emergence or worsening of symptoms close to the next dose of levodopa therapy can be unpredictable if they do not occur regularly. Therefore, in an embodiment, the patient suffering from unpredictable motor fluctuations suffers unpredictable motor fluctuations when end-of-dose motor fluctuations would not be predicted. In preferred examples of this embodiment, the patient suffers from unpredictable motor fluctuations between 30 minutes and 4 hours after the previous dose of levodopa, preferably between 45 minutes and 3 hours after the previous dose of levodopa, more preferably between 1 hour and 2 hours after the previous dose of levodopa.

The treatment of Parkinson's disease with levodopa and opicapone is preferably directed to humans, more preferably adult humans, even more preferably adult humans aged 50 to 80 years.

The Parkinson's disease treated in humans is preferably idiopathic Parkinson's disease.

Symptoms and their Treatment

The trial analysis described in Section D, below, confirms that opicapone is surprisingly effective in reducing absolute off-time in patients with unpredictable motor fluctuations.

In a preferred embodiment, the treatment reduces absolute off-time in a patient suffering from unpredictable motor fluctuations, preferably the treatment reduces absolute off-time by at least 30 minutes per day, more preferably by at least 60 minutes per day. The inventors found that the reduction in absolute off-time is accompanied by an equivalent increase in absolute on-time. Therefore, in another preferred embodiment, the treatment increases absolute on-time in a patient suffering from unpredictable motor fluctuations, preferably the treatment increases absolute on-time by at least 30 minutes per day, more preferably by at least 60 minutes per day. In a more preferred embodiment, the reduction in absolute off-time is accompanied by an equivalent increase in absolute on-time.

As well as reducing absolute off-time, which typically comprises more than 50% end-of-dose motor fluctuations, it is plausible that opicapone can reduce the number of unpredictable “off” periods and/or the time spend suffering from unpredictable motor fluctuations.

In another preferred embodiment, the treatment reduces the time spent suffering from unpredictable motor fluctuations. Preferably the treatment reduces the time spent suffering from unpredictable motor fluctuations by at least 10 minutes per day, more preferably by at least 30 minutes per day, even more preferably by at least 60 minutes per day, compared to patients not treated with opicapone. Additionally or alternatively, the treatment reduces the number of unpredictable “off” periods. Preferably the number of unpredictable “off” periods is reduced by 1 per day, more preferably by 2 per day, even more preferably by 3 per day, compared to patients not treated with opicapone.

Preferably, the unpredictable motor fluctuations treated are selected from the group consisting of severe troublesome dyskinesias, severe/unpredictable motor fluctuations, painful “off” dystonia and morning akinesia. More preferably, the unpredictable motor fluctuations treated are selected from the group consisting of severe/unpredictable motor fluctuations.

The preferred embodiments described above apply equally for the uses of opicapone in the manufacture of a medicament and the methods of treating Parkinson's disease symptoms described at the top of Section B.

For the method of treating the symptoms of Parkinson's disease in a patient suffering from unpredictable motor fluctuations, the method comprises the following steps in the following order:
optionally diagnosing the patient as suffering from unpredictable motor fluctuations; then
administering to the patient a therapeutically effective amount of opicapone, or a pharmaceutically acceptable derivative thereof, in combination with levodopa, or a pharmaceutically acceptable derivative thereof.

### C. Clinical Protocols

### Clinical Trial Design

The results of the two multi-centered double-blinded (DB) randomized placebo-controlled trials BIPARK-I (Ferreira et al., Lancet Neurol., 2016, 15, 154-65) and BIPARK-II (Lees et al., JAMA Neurol., 2017, 74, 197-206) were pooled together. Both clinical trials were performed to evaluate the efficacy and safety of opicapone in addition to levodopa-treated patients with moderate end-of-dose motor fluctuations. All patients had signs of end-of-dose deterioration for at least 4 weeks before screening, with a mean total awake off-time (state of akinesia or decreased mobility) of at least 1.5 hours, excluding morning akinesia. Eligible patients were randomized at baseline to the double-blind phase using a computer-generated scheme (administered by Cenduit, LLC) in a ratio of 1:1:1 to the addition of oral capsular opicapone, 25 mg/day or 50 mg/day, or matching placebo. The double-blind phase assessments occurred at 4-week intervals and the total duration of the double-blind phase could be 14 to 15 weeks. Full details of the inclusion and exclusion criteria and on study design are described in Ferreira et al., Lancet Neurol., 2016, 15, 154-65 and Lees et al., JAMA Neurol., 2017, 74, 197-206, which are incorporated herein by reference. Reductions in the daily dose (but not frequency) of levodopa were allowed between baseline and 3 to 4 weeks after baseline according to the clinical response but were not permitted thereafter.

### Unified Parkinson's Disease Rating Scale

The most widely used clinical scale for assessing the clinical status of patients with Parkinson's disease is the Unified Parkinson's Disease Rating Scale (UPDRS) (Fahn S, Elton RL, UPDRS Program Members. Unified Parkinson's disease rating scale. In Recent Developments in Parkinson's Disease, Vol. 2, eds Fahn S, Marsden C D, Goldstein M. Florham Park, NJ, USA: Macmillan Healthcare Information, 1987:153-63, 293-304). The UPDRS is administered as follows:
Part I: evaluation of mentation, behavior, and mood;
Part II: self-evaluation of the activities of daily life (ADLs) including speech, swallowing, handwriting, dressing, hygiene, falling, salivating, turning in bed, walking, and cutting food;
Part III: clinician-scored monitored motor evaluation;
Part IV: complications of therapy;
Part V: Hoehn and Yahr staging of severity of Parkinson's disease;
Part VI: Schwab and England ADL scale.

Methods for calculation of the total score, as well as analysis of the sub-sections are known to the skilled person.

### Modified Hoehn & Yahr Staging

The Hoehn and Yahr scale is used to describe the progression of Parkinson disease symptoms. The original version (Hoehn M., Yahr M., Neurology, 1967, 17, 427-42) included stages 1 to 5. The modified version includes additional stages 1.5 and 2.5 to allow recording of the intermediate stages of Parkinson's disease.

### Schwab and England Scale

The Schwab and England activities of daily living (ADL) scale is a measure of daily function on a scale of 0 (indicating worst possible function) to 100 (indicating no impairment) (Schwab R., England A., 1969; 152-7).

### Statistical Analysis

Populations undergoing analysis included all randomized patients who took at least 1 dose of study medication and had at least 1 post-baseline off-time assessment. The post-hoc analysis described herein, analyzed Parkinson's disease patients with (“YES”) and without (“NO”) unpredictable on/off fluctuations.

The primary efficacy variable was the change from baseline in absolute off-time. As secondary end-point, the inventors analyzed the absolute on-time as well as the proportions of patients achieving at least a 1-hour reduction in absolute off-time and/or the proportions of patients achieving at least a 1-hour increase in absolute on-time at the end of the double-blind phase, defined as “responders”.

Descriptive statistics of demographic, clinical and therapeutic data were provided for continuous [mean and standard deviation (SD)] and categorical (count and percentage) variables.

Two-group comparisons were performed using Fisher's exact test (categorical variables) and Student's t-test (continuous variables), as appropriate.

Changes from baseline were analysed using ANCOVA with treatment group included as fixed effect factor and the baseline scale score as a covariate. CMH-Test was used to assess differences between OPC dose levels and Placebo taking study into account as a factor.

All P-values reported are two-tailed and a P≤0.05 was considered statistically significant. The software used was SAS 9.4.

### D. Results

### Opicapone Treatment

Opicapone was synthesised as described in WO 2013/089573 and formulated into 25 or 50 mg capsules as described in WO 2010/114405. Study treatment (opicapone or matching placebo) was taken orally once daily in the evening at least 1 hour after the last daily dose of levodopa/DDCI (considered the bedtime dose).

There was no change to the subject's levodopa/DDCI regimen throughout the double-blind period of the study unless adjustment was necessary for subject safety.

Demographic, clinical and therapeutic data of the patients are detailed in Table 1. Considering both trials, 480 patients did not have unpredictable motor fluctuations, while 278 presented unpredictable motor fluctuations during the study. Therefore, this analysis considers approximately one-third of the trial participants.

Demographic, clinical and therapeutic baseline characteristics, namely Hoehn Yahr Stage (HY), Schwab and England ADL Scale, UPDRS part II, UPDRS part III, were comparable both between treatment groups (placebo vs. opicapone (25 mg) and opicapone (50 mg), both separate and pooled data) and between patients with and without unpredictable motor fluctuations (“YES” vs. “NO”), with the exception of: a) a slightly younger age in the placebo group if compared to opicapone (50 mg); b) minor though statistically significant differences of HY, levodopa dose (mg), disease duration and motor fluctuations duration, comparing patients with and without unpredictable motor fluctuations (Table 1). Comparing baseline motor complications presence and severity, the inventors found comparable data among treatment and motor fluctuations groups, except for: a) a slightly higher percentage of on-time, absolute on-time with non-troublesome dyskinesias and a lower percentage of off-time in the placebo group compared to opicapone (25 mg); b) a lower presence of dyskinesias and percentage of on-time with and without troublesome dyskinesias comparing patients with and without unpredictable motor fluctuations within the opicapone (25) mg group (Table 1).

TABLE 1
Baseline clinical characteristics.

| | Placebo | | Opicapone 25 mg | | Opicapone 50 mg | |
|---|---|---|---|---|---|---|
| | YES | NO | YES | NO | YES | NO |
| | N = 93 | N = 162 | N = 98 | N = 143 | N = 87 | N = 175 |
| Age (years)ª | 63.3 ± 9 | 62.6 ± 9.1 | 64.8 ± 8.7 | 62.4 ± 8 ^{c} | 65 ± 9 | 64.4 ± 8 |
| Gender (Male, %) | 51.6 | 57.4 | 67.3 | 56.6 | 55.2 | 62.9 |
| BMI | 27.1 ± 5 | 26.5 ± 4.3 | 27 ± 4.7 | 26.6 ± 4.5 | 27 ± 4.7 | 26 ± 4.3 |
| Disease duration (years) | 8.3 ± 4 | 7.3 ± 3.8 | 9.1 ± 4.7 | 7 ± 3.7 ^{c} | 7.7 ± 4.4 | 7.6 ± 4.2 |
| HY | 2.3 | 2.4 ^{c} | 2.4 | 2.3 | 2.5 | 2.3 ^{c} |
| Schwab and England ADL | 82.9 ± 10.5 | 82.8 ± 12 | 83.1 ± 11 | 83.6 ± 11.1 | 79.5 ± 13.6 | 84.5 ± 10.2 |
| Scale | | | | | | |
| UPDRS-II (Med Off) | 17 ± 7.3 | 18.5 ± 6.8 | 18.4 ± 6.9 | 17.4 ± 7.1 | 19.1 ± 7.2 | 17.5 ± 7.1 |
| UPDRS-III (Med ON) | 24 ± 11.8 | 25.5 ± 12.2 | 24.9 ± 13.6 | 25.2 ± 12.4 | 26.5 ± 14.5 | 24.4 ± 12.5 |
| L-dopa (mg) | 697 ± 320 | 695 ± 323 | 833 ± 400 | 663 ± 335 ^{c} | 747 ± 341 | 673 ± 311 |
| Motor fluctuations | 3 ± 2-3 | 2.3 ± 2^{e} | 3 ± 3 | 2.5 ± 2.3 | 2.6 ± 3 | 2.7 ± 2.8 |
| duration (Years) | | | | | | |
| Absolute “off” time | 6.1 ± 2.2 | 6.1 ± 1.9 | 6.2 ± 2.3 | 6.7 ± 2.1 | 6.4 ± 2.1 | 6.2 ± 1.9 |
| (hours) | | | | | | |
| Percentage “off” time (%)^{b} | 38 ± 14 | 37.6 ± 11.3 | 38 ± 13 | 41.5 ± 13 | 39 ± 11 | 38.6 ± 11.8 |
| Absolute “on” time | 10 ± 2 | 10.1 ± 2 | 9.7 ± 2.2 | 9.4 ± 2.2 | 9.9 ± 2.1 | 9.8 ± 2.1 |
| (hours)^{b} | | | | | | |
| Percentage “on” time (%)^{b} | 62 ± 14 | 62.3 ± 11.3 | 61.2 ± 13.4 | 58.4 ± 12.9 | 60.9 ± 11 | 61.3 ± 11.8 |
| Dyskinesia presence (Yes, | 54.8 | 42.8 | 59.2 | 39.2 ^{c} | 58.6 | 45.7 ^{c} |
| %) | | | | | | |
| Absolute “on” time | 7.5 ± 3.5 | 8 ± 3.2 | 7.5 ± 3.3 | 8 ± 3 | 7.4 ± 3.2 | 8 ± 3 |
| without dyskinesia (hours) | | | | | | |
| Percentage “on” time | 46.7 ± 21.8 | 49.2 ± 20 | 46.5 ± 19.8 | 50 ± 18 | 45.4 ± 19.4 | 50 ± 18 |
| without dyskinesia (%) | | | | | | |
| Absolute “on” time with | 1.9 ± 2.6 | 1.6 ± 2.8 | 1.6 ± 2.2 | 1 ± 2.2 ^{c} | 1.9 ± 2.6 | 1.4 ± 2.4 |
| non-troublesome | | | | | | |
| dyskinesia (hours)^{b} | | | | | | |
| Percentage “on” time with | 11.8 ± 15.9 | 10.2 ± 17 | 10.4 ± 14.1 | 6.6 ± 13.6 ^{c} | 11.9 ± 16.3 | 8.8 ± 14.6 |
| non-troublesome | | | | | | |
| dyskinesia (%) | | | | | | |
| Absolute “on” time with | 0.56 ± 1.4 | 0.46 ± 1.1 | 0.6 ± 1.2 | 0.29 ± 0.8 ^{c} | 0.58 ± 1.1 | 0.36 ± 1 |
| troublesome dyskinesia | | | | | | |
| (hours) | | | | | | |
| Percentage “on” time with | 3.4 ± 8.6 | 2.8 ± 7.3 | 4.1 ± 8.8 | 1.8 ± 5.3 ^{c} | 3.4 ± 6.8 | 2.1 ± 6.5 |
| troublesome dyskinesia | | | | | | |
| (%) | | | | | | |

Values are presented as mean (SD) unless otherwise specified. No statistical significant difference were found comparing: I) opicapone (50 mg) and opicapone (25 mg) vs. placebo; II) opicapone (50 mg) vs. opicapone (25 mg);
“YES” means unpredictable motor fluctuations present; “NO” means unpredictable motor fluctuations absent.
^{a}P < 0.05: opicapone (50 mg) vs. placebo;
^{b}P < 0.05: opicapone (25 mg) vs. placebo;
^{c} P < 0.05; “YES” vs. “NO” within the same treatment group, i.e. placebo, opicapone (25 mg), opicapone (50 mg), separately.

After 14-15 weeks of treatment, among patients with unpredictable motor fluctuations, both opicapone (25 mg) and opicapone (50 mg) were effective in reducing absolute off-time, with a reduction of −61 m and −76 m vs. placebo, respectively ((p<0.01 and <0.001, respectively) (Table 2). Therefore, opicapone is surprisingly effective in difficult-to-treat patients with unpredictable motor fluctuations.

TABLE 2
Changes from baseline to end-point assessment (after 14-15 weeks).

| | | Absolute off-time | Odds Ratio | |
|---|---|---|---|---|
| Group | n | (minutes) | (95% CI) | P-value |
| Total | | | | |
| placebo | 255 | −58.1 | [−78.2, −37.9] | |
| opicapone (25 mg) | 241 | −98.7 | [−118.7, −78.8] | |
| opicapone (50 mg) | 262 | −122 | [−142.5, −101.6] | |
| opicapone (25 mg) vs. placebo | | −40.7 | [−68.3, −13.0] | 0.004 |
| opicapone (50 mg) vs. placebo | | −64 | [−91.6, −36.4] | <0.0001 |
| “YES” | | | | |
| placebo | 93 | −62.9 | [−94.6, −31.2] | |
| opicapone (25 mg) | 98 | −123.8 | [−155.0, −92.7] | |
| opicapone (50 mg) | 87 | −139.3 | [−172.3, −106.3] | |
| opicapone (25 mg) vs. placebo | | −61 | [−104.8, −17.1] | 0.0065 |
| opicapone (50 mg) vs. placebo | | −76.4 | [−121.0, −31.8] | 0.0008 |
| “NO” | | | | |
| placebo | 162 | −53.2 | [−77.3, −29.2] | |
| opicapone (25 mg) | 143 | −73.6 | [−99.5, −47.8] | |
| opicapone (50 mg) | 175 | −104.7 | [−127.8, −81.7] | |
| opicapone (25 mg) vs. placebo | | −20.4 | [−54.7, 13.9] | 0.2435 |
| opicapone (50 mg) vs. placebo | | −51.5 | [−83.9, −19.1] | 0.0019 |

“YES”: Parkinson's disease patients with unpredictable motor fluctuations; “NO”: Parkinson's disease patients without unpredictable motor fluctuations. Significant differences shown in bold.

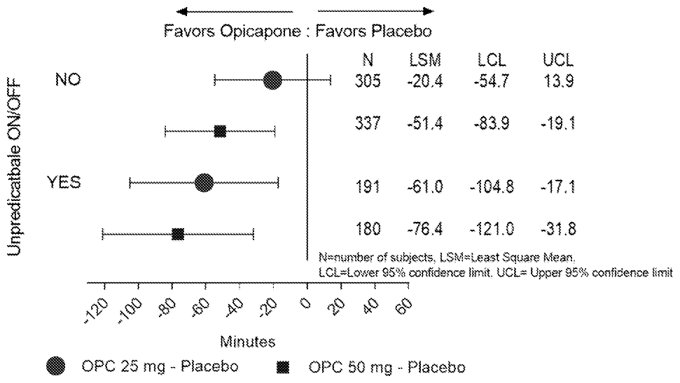

Among patients without unpredictable motor fluctuations, only opicapone (50 mg) resulted in significant absolute off-time reduction (−51 min vs. Placebo; p<0.005) (Table 1; FIG. 1), but at a lower level to that seen in patients with unpredictable motor fluctuations.

The fact that opicapone (25 mg) causes a statistically significant improvement in patients with unpredictable motor fluctuations, but not in those without unpredictable motor fluctuations, it particularly surprising. Not only is the group with unpredictable motor fluctuations smaller (making statistical significance harder to achieve), these patients are at a more advanced stage of disease where a higher dose would be expected to be required. Therefore, opicapone (25 mg) is as effective as opicapone (50 mg) in patients with unpredictable motor fluctuations, yet halves the dose, which reduces cost and could reduce side effects.

Concomitantly, with both opicapone (25 mg) and opicapone (50 mg), the percentage of “responders” for both off-time reduction and on-time increase was significantly higher if compared to placebo for patients with unpredictable motor fluctuations, but only significantly higher with opicapone (50 mg) for patients without unpredictable motor fluctuations (Table 3).

TABLE 3
OFF-time and ON-time Responder Rates (≥1 hour) at Endpoint.

| Group | parameter | responders (%) | P-value |
|---|---|---|---|
| “YES” | | | |
| placebo | off-time reduced | 50.5 | |
| opicapone (25 mg) | off-time reduced | 65.3 | 0.0387 |
| opicapone (50 mg) | off-time reduced | 65.5 | 0.0432 |
| placebo | on-time increased | 48.4 | |
| opicapone (25 mg) | on-time increased | 69.4 | 0.0027 |
| opicapone (50 mg) | on-time increased | 66.7 | 0.0132 |
| “NO” | | | |
| placebo | off-time reduced | 48.1 | |
| opicapone (25 mg) | off-time reduced | 58.7 | 0.0597 |
| opicapone (50 mg) | off-time reduced | 68.6 | 0.0001 |
| placebo | on-time increased | 43.8 | |
| opicapone (25 mg) | on-time increased | 53.8 | 0.0714 |
| opicapone (50 mg) | on-time increased | 61.7 | 0.0011 |

Significant differences shown in bold.

Importantly, opicapone was safe in patients with unpredictable motor fluctuations (Table 4). Dyskinesia was the most frequently reported treatment-emergent adverse events (TEAEs), possibly related to the study drug, with the highest incidence in the opicapone groups (Table 4).

TABLE 4
Treatment-emergent adverse events (TEAEs) reported in at least 5% of
patients in any group in the safety set.

| TEAEs affecting | Placebo | | Opicapone 25 mg | | Opicapone 50 mg | |
|---|---|---|---|---|---|---|
| >5% of pts in any | YES | NO | YES | NO | YES | NO |
| arm | N = 93 | N = 162 | N = 98 | N = 143 | N = 87 | N = 175 |
| Dyskinesias | 7 (7.5) | 9 (5.5) | 25 (25.5) | 14 (9.6) | 19 (21.8) | 35 (19.7) |
| Falls | 5 (5.4) | 7 (4.3) | 8 (8.2) | 3 (2.1) | 5 (5.7) | 3 (1.7) |
| Dry mouth | 1 (1.1) | 2 (1.2) | 8 (8.2) | 8 (5.5) | 3 (3.4) | 5 (2.8) |
| Constipation | 4 (4.3) | 1 (0.6) | 3 (3.1) | 9 (6.2) | 7 (8.0) | 10 (5.6) |
| Insomnia | 3 (3.2) | (0.6) | 6 (6.1) | 11 (7.5) | 3 (3.4) | 6 (3.4) |
| Somnolence | 0 | 5 (3.0) | 7 (7.1) | 3 (2.1) | 2 (2.3) | 3 (1.7) |
| Anxiety | 2 (2.2) | 2.2 (0.8) | 5 (5.1) | 0 | 3 (3.4) | 1.1 (0.4) |
| Nausea | (3.2) | 7 (4.3) | 6 (6.1) | 5 (3.4) | 2 (2.3) | 6 (3.4) |
| Parkinson's disease | 3 (3.2) | 5 (3.0) | 7 (7.1) | 4 (2.7) | 1 (1.1) | 5 (2.8) |
| Headache | 5 (5.4) | 7 (4.3) | 2 (2.0) | 6 (4.1) | 3 (3.4) | 7 (3.9) |
| ↑CPK | 1 (1.1) | 4 (2.4) | 3 (3.1) | 4 (2.7) | 1 (1.1) | 12 (6.7) |
| Hypertension | 0 | 6 (3.7) | 4 ( 4.1) | 6 (4.1) | 1 (1.1) | 7 (3.9) |
| Dizziness | 1 (1.1) | 2 (1.2) | 3 (3.1) | 7 (4.8) | 4 (4.6) | 5 (2.8) |
| Urinary tract | 1 (1.1) | 1 (0.6) | 3 (3.1) | 1 (0.7) | 1 (1.1) | 9 (5.1) |
| infection | | | | | | |
| Weight decrease | 0 | 0 | 0 | 0.1 (0.7) | 1.1 (0.4) | 9 (5.1) |
| Back pain | 2 (2.2) | 5 (3.0) | 1 (1.0) | 4 (2.7) | 2 (2.3) | 1 (0.6) |
| Incidence of any | 9 (3.5) | 9 (3.5) | 6 (2.5) | 7 (2.9) | 10 (3.8) | 13 (4.9) |
| TEAEs leading to | | | | | | |
| Discontinuation | | | | | | |
| Incidence of Serious | 2 (0.8) | 9 (3.5) | 3 (1.2) | 2 (0.8) | 7 (2.6) | 6 (2.3) |
| TEAEs | | | | | | |
| Incidence of Related | 0 | 6 (2.3) | 0.11 (0.4) | 0 | 1 (0.4) | 0 1 (0.4) |
| Serious TEAEs | | | | | | |

Values are expressed as number (%).
CPK: Blood creatine phosphokinase.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A method of treating the symptoms of Parkinson's disease in a patient suffering from unpredictable motor fluctuations, comprising administering to the patient a therapeutically effective amount of opicapone, or a pharmaceutically acceptable derivative thereof, in combination with a therapeutically effective amount of levodopa, or a pharmaceutically acceptable derivative thereof.

2. The method according to claim 1, wherein the opicapone, or a pharmaceutically acceptable derivative thereof, is administered once daily at a dose equivalent to 10 to 100 mg of opicapone.

3. The method according to claim 1, wherein the levodopa, or a pharmaceutically acceptable derivative thereof, is administered 3 to 10 times per day at a total daily dose equivalent to 300 to 2000 mg of levodopa.

4. The method according to claim 1, said combination further comprising a DOPA decarboxylase inhibitor (DDCI).

5. The method according to claim 4, wherein the DDCI is carbidopa or benserazide.

6. The method according to claim 4, wherein the DDCI is administered 3 to 10 times per day at a total daily dose of 25 to 500 mg.

7. The method according to claim 4, wherein the levodopa, or a pharmaceutically acceptable derivative thereof, and the DDCI are administered in a single dosage unit.

8. The method according to claim 1, wherein the patient suffering from unpredictable motor fluctuations is a patient suffering from sudden and random changes from an “on” state to an “off” state over a period of less than 10 minutes with no apparent relationship to the timing of administration of levodopa or a pharmaceutically acceptable derivative thereof.

9. The method according to claim 8, wherein the sudden and random changes from an “on” state to an “off” state (on/off phenomenon) occur over a period of 5 seconds to 5 minutes.

10. The method according to claim 1, wherein the symptom treated is end-of-dose motor fluctuations.

11. The method according to claim 10, wherein the treatment reduces absolute “off”-time by at least 30 minutes per day.

12. The method according to claim 11, wherein the reduction in absolute “off”-time is accompanied by an equivalent increase in absolute “on”-time.

13. The method according to any of claim 1, wherein the treatment reduces the time spent suffering from unpredictable motor fluctuations.

14. (canceled)

15. The method according to claim 1, wherein the patient is diagnosed with Parkinson's disease prior to administering the therapeutically effective amount of opicapone, or a pharmaceutically acceptable derivative thereof, in combination with levodopa, or a pharmaceutically acceptable derivative thereof.

16. (canceled)

17. A method of treating unpredictable motor fluctuations in a patient suffering from Parkinson's disease, comprising administering to the patient a therapeutically effective amount of opicapone, or a pharmaceutically acceptable derivative thereof, in combination with a therapeutically effective amount of levodopa, or a pharmaceutically acceptable derivative thereof.

18. The method according to claim 1, wherein the treatment lasts for at least 10 weeks.

19. The method according to claim 17, wherein the treatment lasts at least 10 weeks.

20. The method according to claim 2, wherein the opicapone, or a pharmaceutically acceptable derivative thereof, is administered once daily at a dose equivalent to 25 to 50 mg of opicapone.

21. The method according to claim 3, wherein the levodopa, or a pharmaceutically acceptable derivative thereof, is administered once daily at a dose equivalent to 500 to 1000 mg of levodopa.

22. The method according to claim 6, wherein the DDCI is administered 3 to 10 times per day at a total daily dose of 75 to 250 mg.
